(19) United States
(12) Patent Application Publication
(10) Pub. No.: US 2009/0007911 A1
(43) Pub. Date: Jan. 8, 2009

(54) Pulmonary Rehabilitation Providing Respiratory Assistance by Application of Positive Airway Pressure

(76) Inventors: Doreen Cleary, Mashpee, MA (US); Scott McGrory, Reading, MA (US)

Correspondence Address: THOMAS P O'CONNELL, 1026A MASSACHUSETTS AVENUE, ARLINGTON, MA, 02476, US

(21) Appl. No.: 12/168,887

(22) Filed: Jul. 7, 2008

Related U.S. Application Data
(63) Continuation-in-part of application No. PCT/US2007/060251, filed on Jan. 8, 2007.
(60) Provisional application No. 60/756,681, filed on Jan. 6, 2006.
(60) Provisional application No. 60/756,680, filed on Jan. 6, 2006.

(51) Int. Cl. A61M 16/00

(52) U.S. Cl. 128/204.17

(57) ABSTRACT

A system for pulmonary rehabilitation by the application of positive airway pressure, characterized in that the system comprises a source of pressurized air, a source of pressurized oxygen, control arrangement for enabling a control of the sources of pressurized air and pressurized oxygen, a source of sterile water, a source of heat, and an output arrangement for providing pressurized and heated air and oxygen to a patient. The system can be rendered portable by use of a mobile casing. Means can be provided for enabling an automatic following of a patient by the casing.

### FIELD OF THE INVENTION

The invention disclosed herein relates generally to rehabilitation systems and methods. More particularly, this disclosure is directed to a system and method for enabling pulmonary rehabilitation, in both gym and homecare settings and during activities of daily living, through the provision of respiratory assistance via “high flow” by the application of positive pressure to the airway of a patient.

### BACKGROUND OF THE INVENTION

In recent decades, Chronic Obstructive Pulmonary Disease (COPD) has become a major cause of morbidity and mortality in the United States. Pulmonary diseases, such as COPD, reduce the ability of one or both lungs to fully expel air during the exhalation phase of the breathing cycle. COPD is commonly characterized by the presence of airflow obstruction due to chronic bronchitis or emphysema. The airflow obstruction in COPD often derives from structural abnormalities in a person's smaller airways. The causes of COPD include inflammation, fibrosis, goblet cell metaplasia, and smooth muscle hypertrophy in the terminal bronchioles. As of 1991, COPD has increased by thirty-three percent (33%) since 1979 to become the fourth leading cause of death in the United States.

The symptoms of Chronic Obstructive Pulmonary Disease include coughing, breathlessness, and wheezing deriving from chronic bronchitis and emphysema. Airflow obstruction limits the patient's airflow during exhalation. The symptoms of COPD progressively worsen over time with sporadic exacerbations often requiring hospitalization. As a result, COPD patients experience a consistently worsening baseline breathing status. Breathlessness tends to be induced at lower and lower levels of effort until it becomes a constant presence. Patients experiencing COPD often are consequently limited in their ability to perform normal tasks and exercises.

Currently, there is no cure for COPD. Prior art treatments for COPD and other respiratory maladies have included efforts to prevent or limit further respiratory damage, pharmacotherapy, and surgery. For example, further respiratory damage has been minimized through the adoption of a healthy lifestyle, such as through a cessation of smoking, regular exercise, and weight control. However, patients seeking to improve pulmonary function are often hampered and even prevented from doing so by the very problems they are seeking to address. Such patients commonly require pulmonary rehabilitation programs including ventilatory muscle training and breathing retraining. Long-term oxygen therapy may also be required.

Pharmacotherapy has included bronchodilators, including beta-agonists, anti-cholinergics, and theophylline, and anti-inflammatories to open up patients' airways as much as possible. Other patients have taken ipratropium bromide or steroids, such as corticosteroids. Furthermore, antibiotics have been employed to prevent infections and influenza. Still further, pheumococcal vaccines are often administered. Unfortunately, there is no evidence that even early, regular use of pharmacotherapy will prevent the progression of COPD.

Surgical intervention has had some success in increasing forced expiratory volume and decreasing total lung capacity thereby improving patients' lung function, dyspnea, and overall quality of life. In one common type of surgery, termed lung volume reduction surgery (LVRS), the most affected portions of a patient's lungs are removed under the theory that the tethering force that tends to keep the intrathoracic airways open was lost in emphysema. By surgically removing the most affected parts of the lungs, the force could be partially restored. Improvements in pulmonary function after surgery have been attributed to at least four possible mechanisms, including enhanced elastic recoil, correction of ventilation/perfusion mismatch, improved efficiency of respiratory musculature, and improved right ventricular filling.

Under the teachings of U.S. Pat. No. 6,258,100, a lung may be collapsed by obstructing an air passageway communicating with the lung portion, such as by placing an obstructing member in the air passageway. Once the air passageway is sealed, the residual air within the lung will be absorbed over time to cause the lung portion to collapse. Under U.S. Pat. No. 6,328,689, a lung constriction device including a sleeve of elastic material is configured to cover at least a portion of a lung. The sleeve has a pair of opened ends to permit the lung portion to be drawn into the sleeve. Once drawn therein, the lung portion is constricted by the sleeve to reduce the size of the lung portion. In other cases, Lung transplantation surgery has been employed in an attempt to combat COPD.

Disadvantageously, surgery, whether in the form of lung volume reduction surgery, lung transplantation, or substantially any other type of surgery, is a highly invasive option that represents an inherent danger to the patient. Furthermore, lung transplantation is often not an option to patients, particularly those with less acute COPD, since lung transplantation requires the corresponding availability of a suitable donor organ.

With the foregoing in mind, it will be appreciated that there is a need for effective methods and systems for combating COPD. More specifically, there is a need for a therapy that is less invasive and less traumatic than LVRS and that is capable of providing more permanent results than pharmacotherapy in enabling improved breathing in patients thereby to permit exercise and participation in daily activities.

### SUMMARY OF THE INVENTION

The present invention is founded on the basic object of providing a pulmonary rehabilitation system and method applicable in home and health care settings that can enable persons suffering from chronic obstructive pulmonary disease and other pulmonary maladies to engage in rehabilitation, exercise, and, in certain embodiments, everyday activities in an efficient and comfortable manner.

A more particular object of embodiments of the invention is to provide a pulmonary rehabilitation system and method applicable in home and health care settings that enable increased mobility for those suffering from pulmonary disease.

A related object of embodiments of the invention is to provide a pulmonary rehabilitation system and method that enable increased activity and exercise for those suffering from pulmonary disease.

Another particular object of embodiments of the invention is to provide a pulmonary rehabilitation system and method that enable an adjustment of air flow rates, oxygenation, air temperature, and other properties to accommodate varied circumstances and to provide comfort and ease of respiration to a user.

Still another object of particular embodiments of the invention is to enable an improvement of a patient's pulmonary condition without resort to invasive operative and other procedures.

These and further objects and advantages of embodiments of the invention will become obvious not only to one who reviews the present specification and drawings but also to one who has an opportunity to make use of an embodiment of the instant invention for a pulmonary rehabilitation system and method applicable in the home and health care setting, including during activities of daily living. However, it will be appreciated that, although the accomplishment of each of the foregoing objects in a single embodiment of the invention may be possible and indeed preferred, not all embodiments will seek or need to accomplish each and every potential object and advantage. Nonetheless, all such embodiments should be considered within the scope of the present invention.

### BRIEF DESCRIPTION OF THE DRAWINGS

In the accompanying drawings:

FIG. 1 is a schematic view of a pulmonary rehabilitation system pursuant to the present invention;

FIG. 2 is a schematic view of an embodiment of a mobile pulmonary rehabilitation system according to the instant invention;

FIG. 3 is a schematic view of an embodiment of a pulmonary rehabilitation system for use in conjunction with an exercise program according to the invention disclosed herein;

FIG. 4 is a perspective view of a mobile pulmonary rehabilitation system according to the present invention in a waist pack configuration;

FIG. 5 is a perspective view of the mobile pulmonary rehabilitation system of FIG. 4 in a partially disassembled condition;

FIG. 6 is a perspective view of a mobile pulmonary rehabilitation system as disclosed herein in a should pack configuration;

FIG. 7 is a perspective view of the mobile pulmonary rehabilitation system of FIG. 6 in an opened condition;

FIGS. 8 and 9 are photographs of a patient receiving pulmonary rehabilitation from a pulmonary rehabilitation system as taught herein;

FIG. 10 is a photograph of an oxygen flowmeter for use under the present invention;

FIG. 11 is a photograph of a compressed air flowmeter usable with the invention disclosed herein;

FIG. 12 is a photograph of a nasal cannula for use in pulmonary rehabilitation as taught herein;

FIG. 13 is a photograph of a column humidifier as used under the instant invention;

FIG. 14 is a photograph of a heating/humidifying system usable pursuant to the pulmonary rehabilitation system;

FIG. 15 is a photograph of a connection arrangement as taught herein;

FIG. 16 is a photograph of a further connection arrangement as taught herein;

FIG. 17 is a photograph of a large bore conduit for use under the present invention;

FIGS. 18 and 19 is a photograph of a cannula arrangement;

FIGS. 20-24 are photographs of connectors usable under the present invention;

FIG. 25 is a photograph of a heating/humidifying system under the present invention;

FIGS. 26-28 are photographs of an air-oxygen blending arrangement for use under the instant invention;

FIG. 29 is a perspective view of another embodiment of a pulmonary rehabilitation system under the present invention;

FIG. 30 is a view in rear elevation of the system of FIG. 29;

FIG. 31 is a partially sectioned view of a system under the instant invention;

FIG. 32 is a top plan view of a control panel;

FIG. 33 is a partially sectioned view of a further system under the instant invention;

FIG. 34 is a partially sectioned view of yet another portable pulmonary rehabilitation system as taught herein;

FIG. 35 is a view in front elevation of a control and access panel;

FIG. 36 is a top plan view of a further control and access panel for use in relation to a pulmonary rehabilitation system;

FIG. 37 is a partially sectioned view of a further portable pulmonary rehabilitation system as taught herein;

FIG. 38 is a perspective view of a hands free walker attachment;

FIG. 39 is a perspective view of a “follow me” arrangement for use under the invention disclosed herein;

FIG. 40 is a perspective view of a further mobile pulmonary rehabilitation system according to the present invention; and

FIG. 41 is a perspective view of the mobile pulmonary rehabilitation system of FIG. 40 in a partially disassembled form.

### DETAILED DESCRIPTION OF PREFERRED EMBODIMENTS

As is the case with many inventions, the present invention for a system and method for pulmonary rehabilitation by the application of positive airway pressure is subject to a wide variety of embodiments. However, to ensure that one skilled in the art will be able to understand and, in appropriate cases, practice the present invention, certain preferred embodiments of the broader invention revealed herein are described below and shown in the accompanying drawing figures. Before any particular embodiment of the invention is explained in detail, it must be made clear that the following details of construction, descriptions of geometry, and illustrations of inventive concepts are mere examples of the many possible manifestations of the invention.

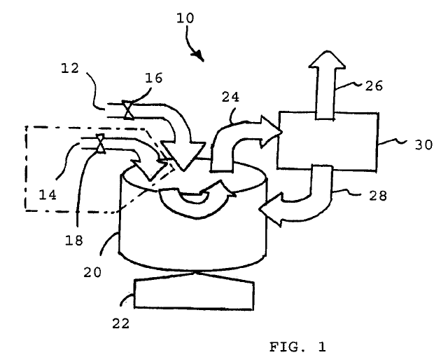

Looking more particularly to the drawings, a basic system for pulmonary rehabilitation by the application of positive airway pressure is depicted schematically in FIG. 1 where the system is indicated generally at 10. The pulmonary rehabilitation system 10 has a pressurized air input 12 that combines with a pressurized oxygen input 14. The flow of air can be controlled by an air input control valve 16, and the flow of oxygen can be controlled by an oxygen input control valve 18. The flow of air and the flow of oxygen can be varied individually and proportionally as will be described further hereinbelow. In one example of the invention, the flow of air can be varied between approximately 0 and approximately 40 liters per minute (L/M) and the oxygen flow can be varied between approximately 5 and approximately 37 L/M.

The pressurized air, the pressurized oxygen, or, if after the two are combined, the combination thereof can be humidified by any effective method with a volume of sterile water 20. A heat source 22 can be applied to the pressurized air, the pressurized oxygen, or the combination thereof to bring it to an elevated temperature condition. In one specific practice of the invention, the heat source 22 can raise the gas to a temperature of, for example, 33.5 degrees Celsius. An output conduit 24 can be fluidically connected to an output connection 30, which can be coupled to a patient cannula 26 or other means for imparting the positive pressure provided by the pulmonary rehabilitation system 10 to an airway of a patient. A condensation return 28 can be provided in certain embodiments for returning condensed fluid to the volume of water 20.

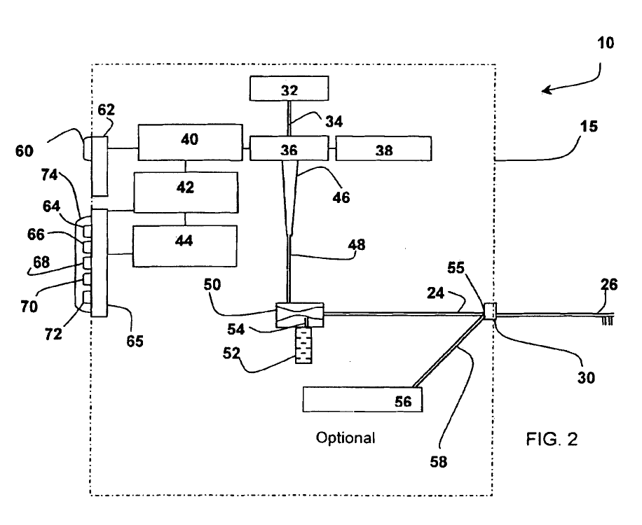

The pulmonary rehabilitation system 10 and the methods by which the same can be used can pursue widely varied embodiments. In one manifestation of the invention, for example, the pulmonary rehabilitation system 10 can be constructed for mobile usage to provide, among other things, increased mobility to persons who could be aided by the positive airway pressure provided by the present invention. One example of such a manifestation of the invention is depicted schematically in FIG. 2 where components of the pulmonary rehabilitation system 10 are retained by a mobile casing 15. The mobile casing 15 can, by way of example, take the form of a waist pack, a backpack, a shoulder pack, rolling backpack, or any other readily portable arrangement. The mobile casing 15 could comprise a mesh casing, a plastic casing, or any other type of mobile casing or combination thereof.

Air can be drawn into the pulmonary rehabilitation system 10 of FIG. 2 through a filter 32, which can comprise a HEPA filter or any other suitable filter 32, by operation of a fan 36 that is operated by a fan motor 38. The fan motor 38 can be powered by a power source 40, which can be a primary power source. In one embodiment of the invention, the fan motor 38 can comprise a DC brushless motor and the fan 36 can comprise a set of turbofan blades. Both can be disposed inline with the filter 32. A secondary power source 42 can provide an alternative source of power, and a power source selector 72 can enable a manual and, additionally or alternatively, an automatic selection between the primary and secondary power sources 40 and 42.

A power indicator/alarm 62 can provide a visual, audible (i.e., “Battery Low” or the like), tactile, or any other type of indication of the power remaining in either or both power sources 40 and 42. The power indicator/alarm 62 can additionally enter an alarm condition 20 when either or both power sources 40 and 42 falls below a predetermined minimum power reserve. The primary and secondary power sources 40 and 42 can be rechargeable by use of a charging port 60 or any other effective arrangement. The primary and secondary power sources 40 and 42 could each comprise, for example, one or more lithium batteries, which could be rechargeable by use of a 110 V AC/60 Hz home charger or a 12 V DC mobile charger.

Air can reach the fan 36 from the filter 32 either directly or through a conduit 34, which can comprise, by way of example, a 2-inch tube configured inline with the fan 36. Of course, it will be appreciated that the order of these and other components in the pulmonary rehabilitation system 10 could be readily interchanged. For example, the fan 36 could readily drive air through the filter 32 instead of drawing the air therethrough. Air can be driven through a compression conduit 46 that can narrow in cross sectional area to produce a compression of the air driven therethrough. In one embodiment, the compression conduit 46 can comprise a flexible tube of annular cross section that can narrow as it winds through the mobile casing 15 to a diameter of 0.125 inches over a 20-inch length of conduit.

Compressed, the air then travels through heated conduit 48 to bring the air to an elevated temperature condition. The heated conduit 48 can, for example, comprise one or more heated stainless steel tubes. Now heated, the compressed air can be passed through a venturi chamber 50 with a portion of reduced cross section. A water conduit 54 can be disposed in the venturi 50, such as just after the portion of reduced cross section, to enable the creation of a relative vacuum of air by a venturi effect. A water supply 52 can be disposed in fluidic association with the water conduit 54 such that water can be drawn through the water conduit 54 and into the stream of air thereby misting and humidifying the compressed, heated air. The water supply 52 can be of any suitable type. In certain embodiments, the water supply 52 can comprise a removable and replaceable container of water, such as a 2-ounce vial of sterile water.

The heated, compressed, and humidified air can, in certain practices of the invention, be infused with a volume of compressed oxygen from an oxygen source 56, such as an oxygen tank, a wall outlet, or any other source. In the depicted embodiment, an oxygen supply conduit 58 is coupled to an output hose 24 from the venturi 50 at a Y connector 55. An output branch of the Y connector 55 can be fluidically coupled to an air output connector 30, which can be disposed to an exterior portion of the mobile casing 15. The output connector 30 can in turn be adapted for removably and replaceably coupling to a patient air supply arrangement, which in this case comprises a nasal cannula 26. Other air supply arrangements are, of course, possible and well within the scope of the present invention.

A control interface 65 can be retained relative to the mobile casing 15 to enable a control of certain variables during operation of the pulmonary rehabilitation system 10. A system control unit 44 is operably associated with the control interface 65, the power sources 40 and 42, and the remaining components of the pulmonary rehabilitation system 10. The volume of air driven through the system 10 can be selectively varied by use of an air volume control 64 to control the operation of the fan 36. The oxygen supply can be adjusted by operation of an oxygen supply control 66 to control output from the oxygen source 56. The humidity added to the flow of air from the water supply 52 can be manipulated by use of a humidity control 68. Still further, a heat control 70 can adjust the temperature condition of the volume of air as affected by the heated conduit 48. A lid 74, which can be clear, can be provided to shield the control interface 65.

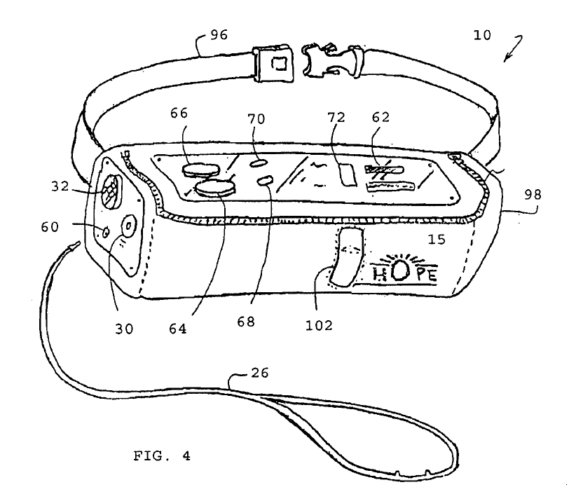

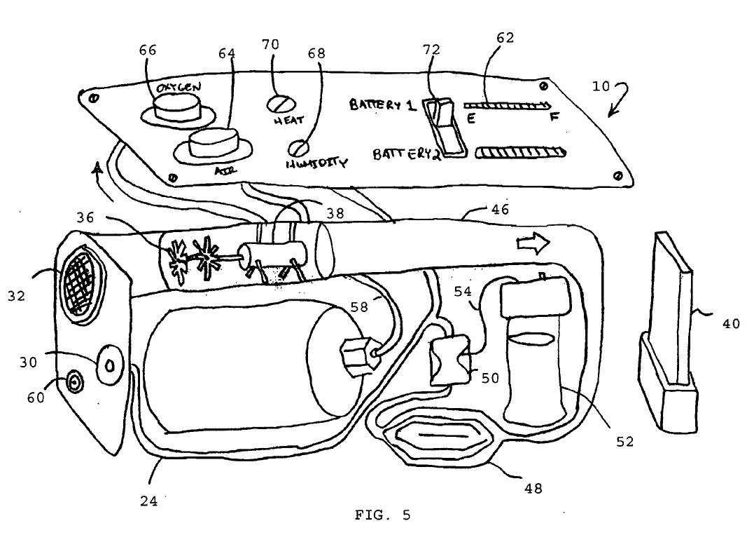

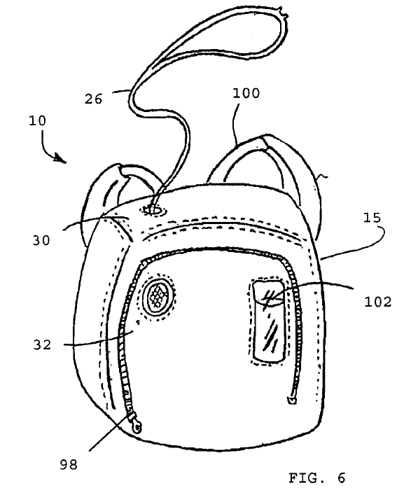

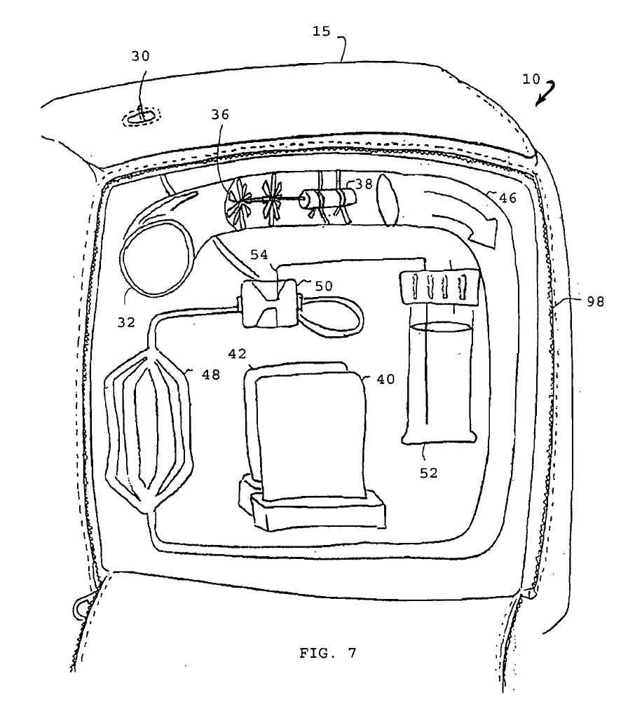

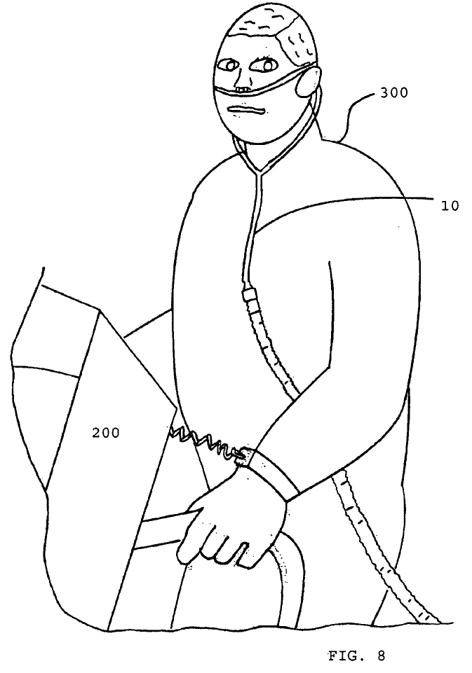

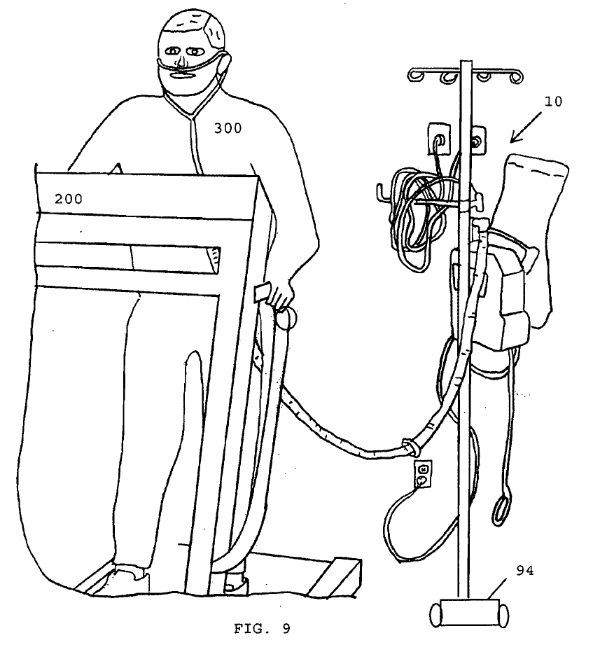

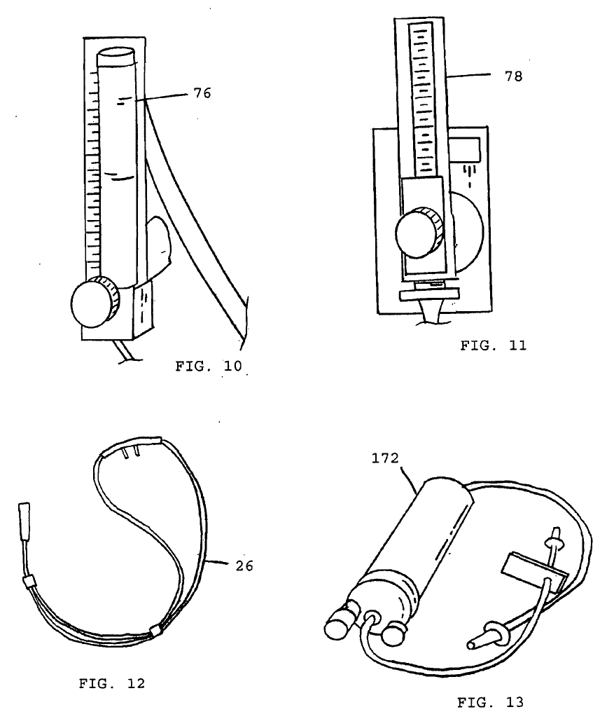

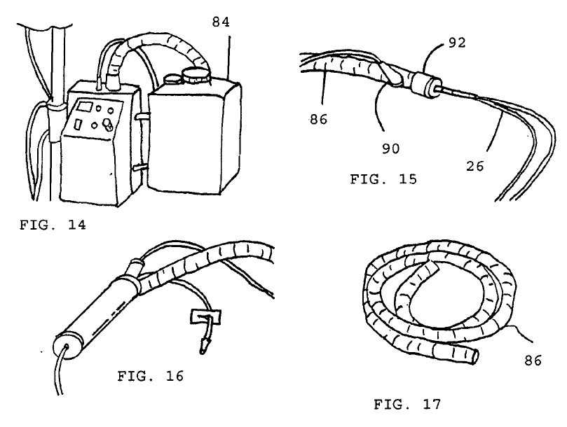

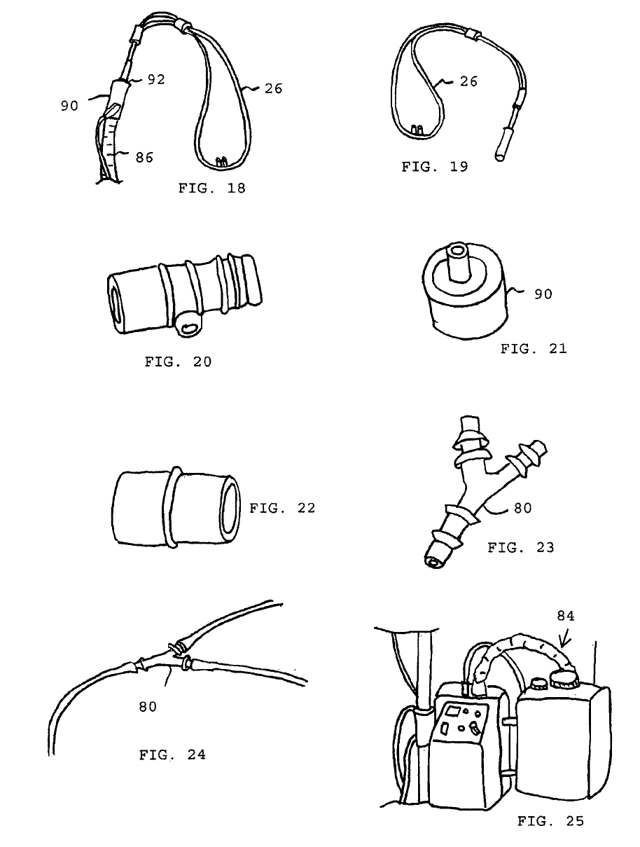

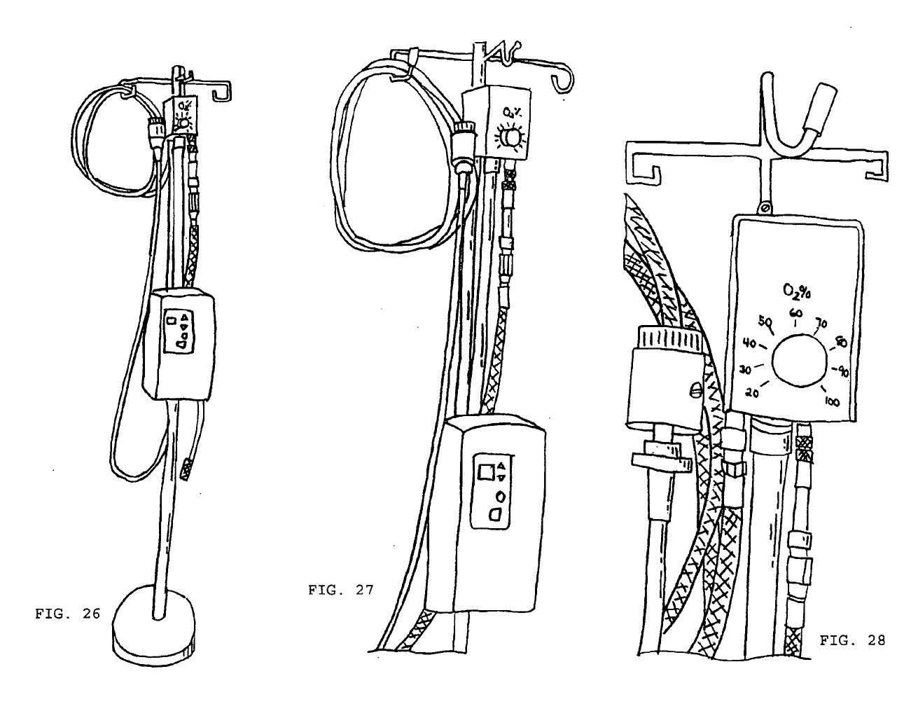

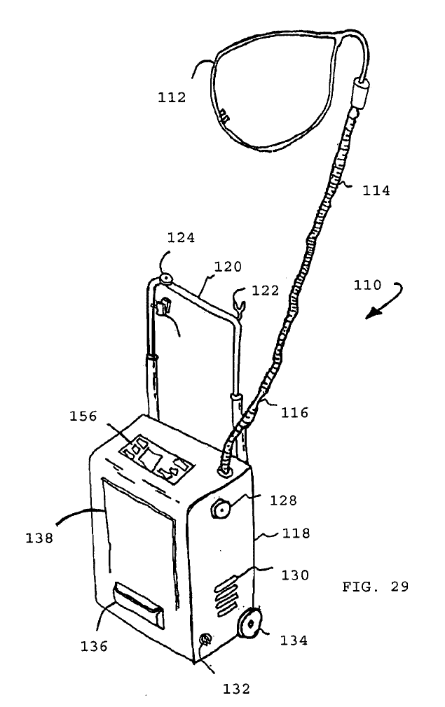

One mobile version of the pulmonary rehabilitation system 10 is depicted in FIGS. 4 and 5, where the pulmonary rehabilitation system 10 is configured as a waist pack. There, a belt arrangement 96 can be employed to retain the mobile unit 15 about the waist of a patient. Access to the interior components of the pulmonary rehabilitation system 10 can be had by operation of a zipper 98. A water level indicator window 102 can be provided in the shell of the mobile unit 15 to enable a viewing of a water level of the water supply 52. The charging port 60, the output connector 30, and the inlet filter 32 are disposed to a side of the mobile unit 15 while the air volume control 64, the oxygen supply control 66, the humidity control 68, the heat control 70, the power source selector 72, and the power indicator/alarm 62 can be disposed to a top of the mobile unit 15.

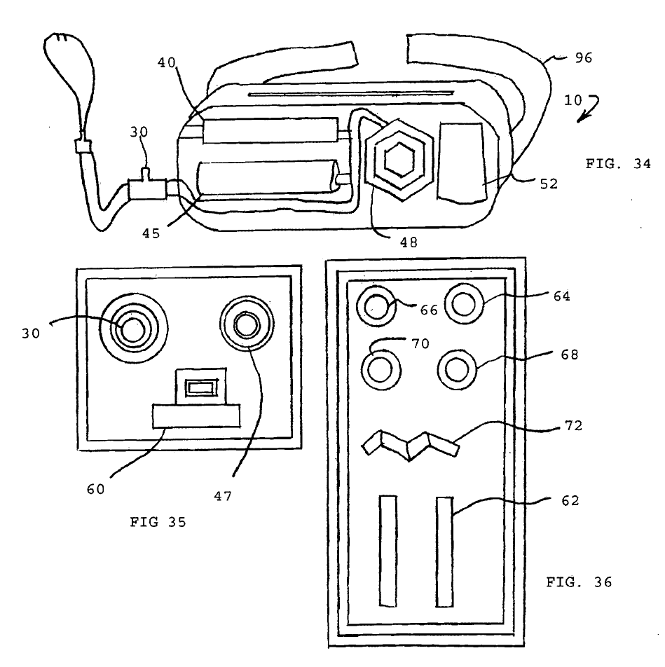

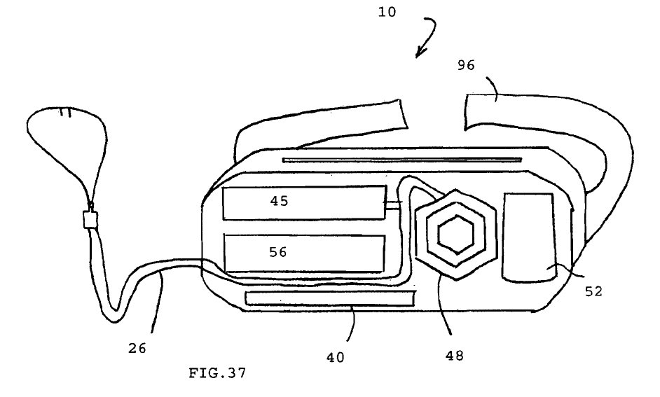

FIGS. 34 through 37 again depict a mobile version of the pulmonary rehabilitation system 10 in belt form. A belt 96 is again provided for being worn about a patient's waist. In FIG. 34, a battery 40 provides power to the system 10. A compressor 45 is employed for compressing gases within the system 10. An insulated heater 48 heats compressed gases provided by the compressor 45, and a water tank 52 provides water to hydrate air supplied to a patient. An oxygen port 30 is provided for enabling a titration of oxygen inline to the nasal cannula thereby to enable a provision of heated, humidified, and compressed air titrated with oxygen. Control panels for the system 10 are shown in FIGS. 35 and 36 again with similarly employed reference numerals with the addition of an air meter 47. In FIG. 37, an oxygen tank 56 is additionally provided.

In FIGS. 6 and 7, the pulmonary rehabilitation system 10 takes the form of a backpack. Backpack straps 100 are provided for retaining the mobile unit 15 relative to a user's shoulders. The water level indicator window 102 and the filter 32 are disposed to an exterior side of the mobile unit 15. An air output connector 30 is disposed to a top side of the mobile unit 15 such that the cannula 26 would be readily available to a user of the pulmonary rehabilitation system 10. A zipper 98 can again be provided for enabling access to the interior components of the pulmonary rehabilitation system 10.

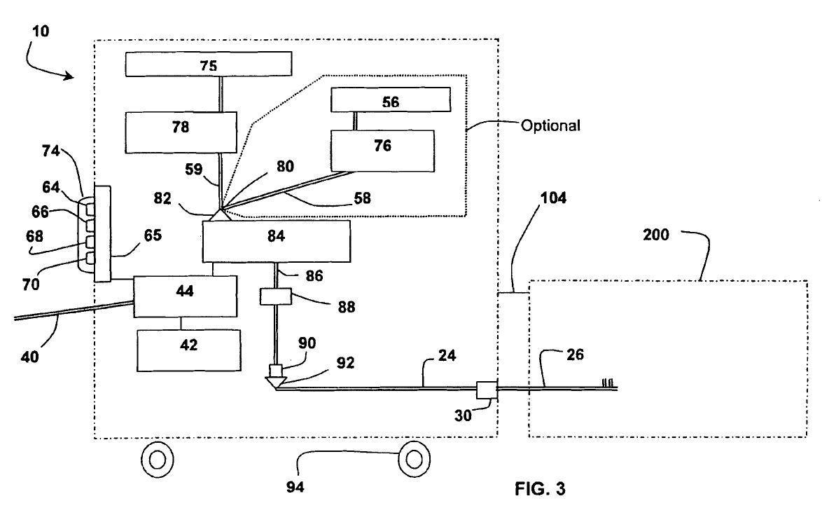

Alternative embodiments of the pulmonary rehabilitation system 10 can be configured for use in a less mobile manner, such as might be used by a patient 300 in a pulmonary rehabilitation program on, by way of example, a treadmill, a stair machine, a stationary bicycle, or any other exercise device 200. Such an embodiment of the pulmonary rehabilitation system 10 is depicted schematically in FIG. 3 and in photographs in, for example, FIGS. 8 and 9. As FIG. 3 depicts, air can be provided by a compressed air source 75, which can be an air compressor, a wall source, an air tank, or any other source of compressed air. Similarly, compressed oxygen can be provided by an oxygen source 56, which can comprise an oxygen tank, a wall source, or any other source of oxygen.

An oxygen flowmeter 76 can measure and regulate the flow of oxygen from the oxygen source 56 while a compressed air flowmeter 78 can measure and regulate the flow of compressed air from the compressed air source 75. The oxygen flowmeter 76 can in certain embodiments be calibrated to regulate flow between 1 and 75 L/M and can be plugged into a 50 psi oxygen wall outlet. The compressed air, which can also be provided by a 50 psi oxygen wall outlet, travels from the compressed air flowmeter 78 through a compressed air conduit 59. The compressed air conduit 59 couples to a first branch of a Y connector 80. The compressed oxygen travels from the oxygen flowmeter 76 through an oxygen supply conduit 58, which couples to a second branch of the Y connector 80.

The outlet branch of the Y connector 80 is fluidically associated with a heating/humidifying system 84 by use of a cone adaptor 82. The heating/humidifying system 84 can be provided as a stand alone arrangement or it can be assembled from necessary components as in the mobile embodiment of FIG. 2. One knowledgeable in the art will be aware that prior art heating/humidifying systems 84 have been disclosed that are essentially self-contained. One such heating/humidifying system 84 is sold under the trademark CONCHA by Hudson Respiratory Care, Inc.

Heated and humidified, the compressed air and oxygen gas can pass through a large bore conduit 88. A thermometer port 88 can be fluidically associated with the large bore conduit 88 to enable a sensing of the gas temperature. The large bore conduit 88 can be coupled to a cone adaptor 92 by an adaptor 90. In turn, the cone adaptor 92 can be coupled to an output conduit 24. The output conduit 24 can couple to an output connector 30, which can removably and replaceably engage a nasal cannula 26 or any other arrangement for providing the heated, humidified, and oxygenated gas to a patient.

A control interface 65 can again be retained relative to the pulmonary rehabilitation system 10 to enable a control of certain variables during operation thereof, and a system control unit 44 can be operably coupled with the control interface 65 and the remaining components of the pulmonary rehabilitation system 10. A power source 40 in the present embodiment can comprise a power cord, which could be supplemented by a secondary, battery-type power source 42 where necessary or desirable. The volume of air driven through the system 10 can be selectively varied by use of an air volume control 64 to control the compressed air flowmeter 78. The oxygen supply can be controlled by operation of an oxygen supply control 66 to control the oxygen flowmeter 76. The humidity added to the flow of air by the heating/humidifying system 84 can be manipulated by use of a humidity control 68. Still further, a heat control 70 can adjust the temperature condition of the volume of air as affected by the heating/humidifying system 84. Again, a lid 74 can be provided to shield the control interface 65.

The overall pulmonary rehabilitation system 10 can be disposed on wheels 94 or any other arrangement for enabling a portability of the system 10. In certain embodiments, the wheels 94 can comprise the wheels of an IV stand, and the pulmonary rehabilitation system 10 can be retained relative thereto. The pulmonary rehabilitation system 10 can thus be used in varied locations and, where desirable, in relation to various exercise devices 200, such as treadmills, stair climbers, stationary bicycles, and any other type of exercise device 200.

An alternative portable pulmonary rehabilitation system is indicated at 110 in, for example, FIGS. 29, 30, and 31. There, the system 110 has a nasal cannula 112 fluidically coupled to a case 118 by an thermally insulated tube 114. Interposed along the tube 114 is what can be termed a weak link disconnect 116, which can allow the tube 114 to separate when necessary. The case 118 has a telescoping handle 120 coupled thereto. A cannula resting hook 122 enables a retention of the nasal cannula 112 during periods of non-use. A ramp flow control 124 is provided for controlling air output. The ramp flow control 124 can operate, for example, by enabling a depressing of a button to ramp up air flow, such as by increments of 5-10 lpm. Flows can be preset as prescribed by a physician.

An oximeter 126 is operably associated with the system 110 for enabling a testing of oxygen levels in a patient's blood. An oxygen control knob 128 is retained on the case 118, and vents 130 allow a flow of air into and out of the open inner volume of the case 118.

Power for the system 110 can be provided by a battery 148, such as a Li-Ion battery. External power for powering the system 110 and, possibly, for recharging the battery 148 can be provided through a power cord 132. The system 110 can have wheels 134 rotatably attached to the case 118. In certain embodiments, one or both wheels 134 can be lockable to fix the system 110 against inadvertent movement. The case 118 can have a pouch 136 for enabling a storage of personal items, attachments, replacement components, and the like. A closure flap 138 can enable assess to the open inner volume of the case 118.

As FIG. 31 shows, the system 110 can retain a volume of sterile water in a water compartment 144. Oxygen can be retained in an oxygen canister 142, and a compressor 146 can operate to compress fluids in the system 110. Power can be provided by a rechargeable battery 148. Water and oxygen can pass through a venturi 140 prior to being dispensed to the patient through a hose 114 leading to a cannula 112.

In the alternative construction of the system 110 of FIG. 33, first and second batteries 148A and 148B can provide power. A compressor 146 and an oxygen tank 142 are fluidically associated with a heater humidifier 172. An oxygen connector 176 can enable a secondary exchange of fluids. A purge valve and collection bag arrangement 174 can be interposed along the insulated tube 114. Presets 155, such as parameters established by a doctor, can be provided for use of the system 110.

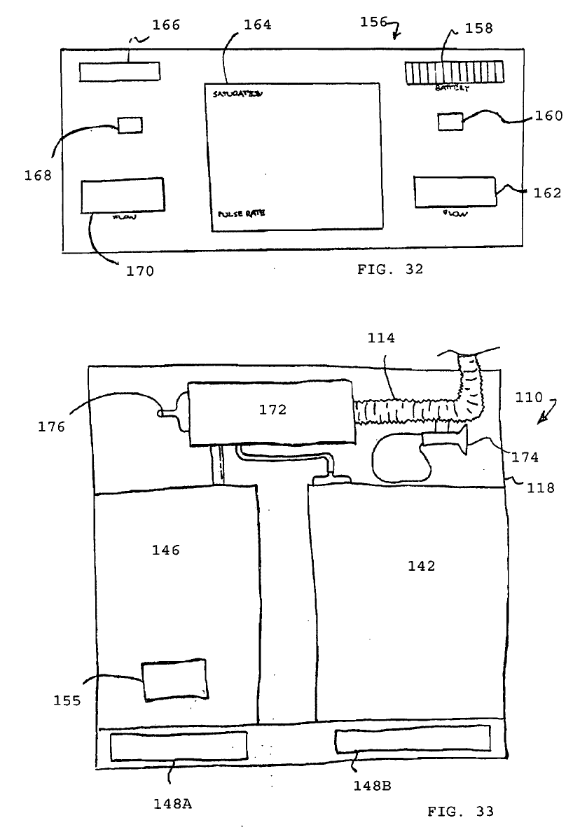

A control panel 156 for the system 110 is shown in FIG. 32. The control panel 156 has a battery indicator 158, a humidity regulator 160, an oxygen flow rate control 162, an oximeter display 164, a pedometer 166, a heat regulator 168, and a compressed air flow rate control 170. The control panel 156 can be suitably located on the case 118, such as by being at the top thereof as in FIG. 29. Audible and visual alarms can be built into the oximeter display 164 and the battery display 158 for providing alarms regarding variances from predetermined operating conditions.

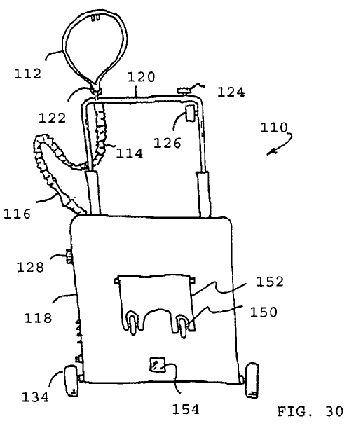

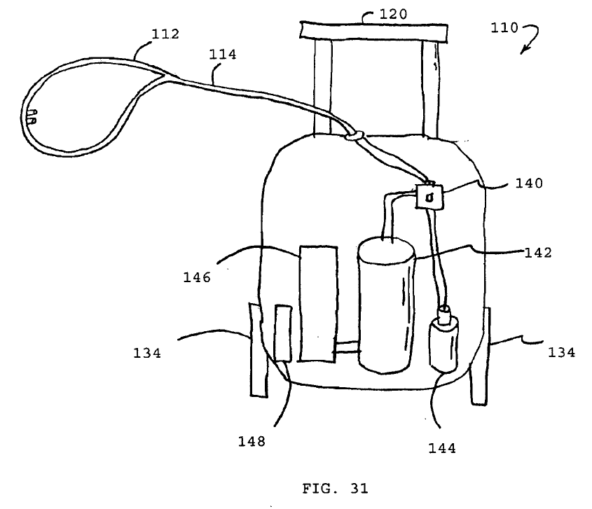

With combined reference to FIGS. 30 and 39, one sees that the system 110 can include secondary wheels 150 attached to the case 118 by a pivoting wheel mount 152. Under this construction, the system 110 can be most stably supported by pivoting the pivoting wheel mount 152 away from the body of the case 118 thereby to provide multiple points of contact with a ground surface.

In a further aspect of the invention, a “follow me” transceiver 154 can be operably associated with the case 118, and a “follow me” member, such as an ankle band 188, with a “follow me” transponder 192 disposed thereon can be retained by a patient. The battery 148 can provide power to a propulsion system. By means known to those skilled in the art, therefore, the case 118 can be programmed to follow a patient automatically with no need for a physical pushing or pulling of the case 118 by the patient.

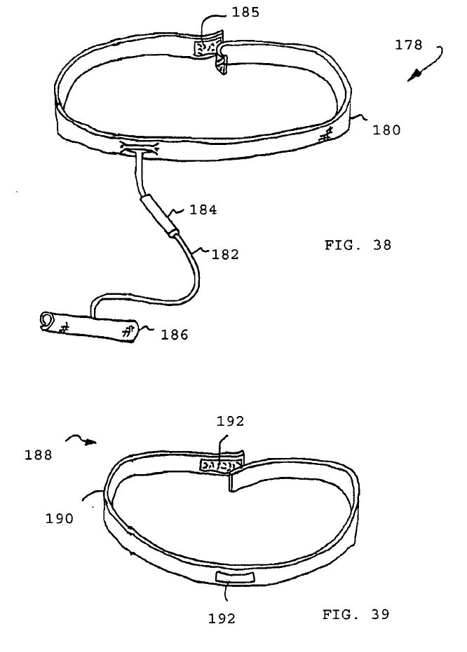

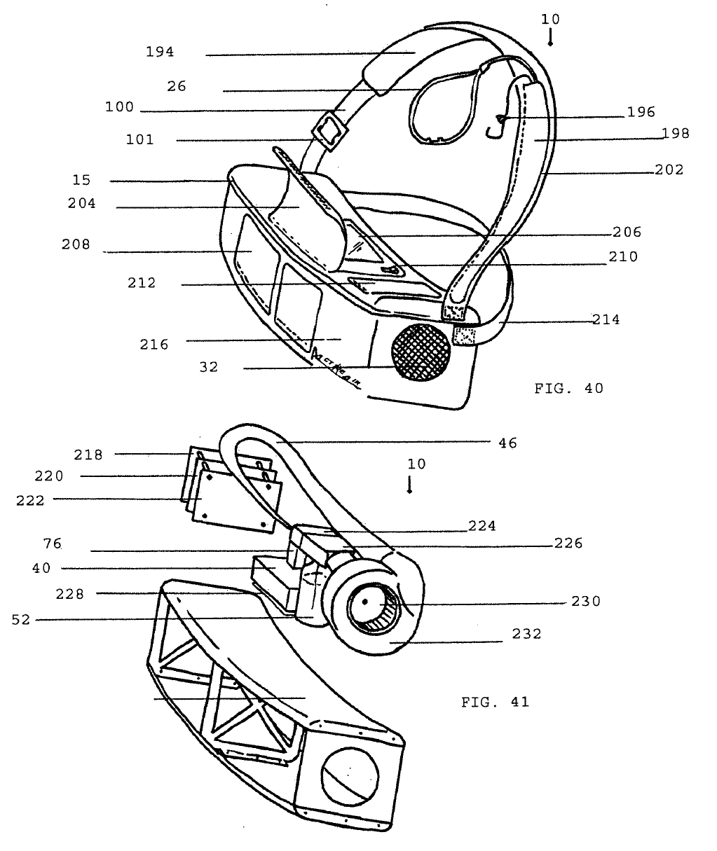

A lower technology hands-free arrangement is shown in FIG. 38. There, a belt 180 with a hook and loop 185 or other fastening arrangement is provided for being worn by a patient. A tether 182 has a first end fixed to the belt 180 and a second end fixed to a handle attachment 186 that can be employed to couple to the handle 120 of the system 110, such as by a hook and loop arrangement. A resilient member 184 can be interposed along the tether 182 to enable smooth a towing of the case 118 as a patient walks with the belt 180 disposed around his or her waist.

As FIGS. 8 and 9 show, the pulmonary rehabilitation system 10 can be employed to provide respiratory assistance to a patient 300, such as a patient with chronic obstructive pulmonary disease (COPD), by the application of positive airway pressure through the nasal cannula 26 or other arrangement. More particularly, it has been found that the pulmonary rehabilitation system 10 can decrease a patient's shortness of breath by opening his or her airway by operation of a high flow/pressure system 10 that emits filtered, heated, humidified, oxygenated, and compressed air. The pulmonary rehabilitation system 10 can, therefore, enable the patient to exercise at increased levels and for increased periods of time as compared to the patient's ability to do so without the pulmonary rehabilitation system 10.

Where the pulmonary rehabilitation system 10 is mobile as in the embodiments of, for example, FIGS. 2 and 4-7, the system 10 can decrease shortness of breath by opening the patient's airways with filtered, heated, humidified, and oxygenated air while the patient is ambulating and during activities of daily living. Mobile versions of the pulmonary rehabilitation system 10 are self contained, highly portable, and comfortable to wear. Furthermore, while air is advanced through multiple stages, the system 10 outputs a smooth and continuous stream of air for the patient.

Since the emitted air is heated and humidified, there will not be a drying effect on the patient's nasal cavity. Also, by exploiting the ability of the system 10 to adjust air pressure, a therapist, a patient, or other user can decrease the patient's shortness of breath by increasing the pressure in the patient's airway to keep the airway open during a pulmonary rehabilitation exercise session. Furthermore, with the ability to adjust the oxygen content of the emitted air, a user can adjust oxygen as necessary or desirable to maintain adequate oxygen saturation. In certain embodiments, an oximeter with automatic biofeedback adjustment can be built into the pulmonary rehabilitation system 10.

Still further, consumable components of the pulmonary rehabilitation system 10, whether in the mobile version or what can be termed the exercise device version, can be readily exchanged. For example, the heating/humidifying system 84 and the components thereof can be disposable and readily replaceable. Also, pre-filled, disposable sterile water containers can be employed as the water supply 52. Even further, the filter 32 can be readily removed, disposed of, and replaced. Even further, where an oxygen tank is employed as the oxygen source 56, the tank can be readily removed and replaced when spent. Similarly, should one or both batter power sources 40 and 42 need replacement, a user need only open the mobile unit 15 to access the same.

During usage of the pulmonary rehabilitation system 10, whether in relation to an exercise device 200 or otherwise, the patient 300 is instructed to breathe the air emitted by the nasal cannula 26 in through his or her nose while keeping his or her mouth shut. Such a practice will create a positive airway pressure thereby to tend to keep the patient's airways open. Where a patient 300 has difficulty keeping his or her mouth closed, a higher flow of air can be provided to increase patient comfort. In practice, the positive air pressure and rehabilitation provided by the pulmonary rehabilitation system 10 may be administered pursuant to the instruction and prescription of a medical doctor who has ideally received training through a pulmonary rehabilitation program. Preliminary, subsequent, or additional rehabilitation may be provided in supplementation of pulmonary rehabilitation programs exploiting the present invention.

Still another embodiment of the pulmonary rehabilitation system 10 is shown in FIGS.>40 and 41. There, the system 10 has a mobile casing 15 that is slightly curved in shape to form fit a patient's waist. The casing 15 can be approximately 15 cm×15 cm×30 cm in one preferred embodiment. A skeleton 25 is is covered with a 600D polyester fabric shell 216 that has a padded backing for maximum comfort. The polyester fabric shell 216 is backed with a semi rigid vinyl sheet for protection of the more critical components while ensuring comfort during use.

The weight of the mobile unit 15 is supported by an adjustable shoulder strap 100 that can be worn diagonally across a patient's body. A wide, padded upper section 194 distributes the weight of the mobile unit 15. The lower rear portion of the shoulder strap 100 has an adjustable buckle 101 while the front of the strap 100 has a hollow sleeve 198 that is thermally insulated. This design helps keep the wires and cannula tubes 26 hidden for aesthetic reasons, eliminates tangles, increases ease of mobility, and aids in minimizing condensation and heat loss within the cannula tubing 26. The hollow sleeve 198 also houses an oximeter ear clip lead 196, an infrared humidity/heat sensor 195 for sensing the properties within the cannula tubes 26, and the cannula tubing 26. The hollow sleeve 198 is accessible via a hook and loop fastening arrangement 202 along the entire length of shoulder strap 100 to aid in changing the cannula tubing 26 as necessary. To stabilize the mobile unit 15 when the patient is moving, walking, or running, the mobile unit 15 can additionally be secured at the patient's hip with a wide nylon adjustable waist strap 214 and a large plastic snap-buckle (not shown). When so secured, the wide strap 214 maximizes comfort during movement. The large snap buckle ensures ease of use for physically challenged patients. Several fabric covered doors secured via hook and loop fasteners and hinged on double stitched seams are available on the exterior of the mobile unit 15 to allow access to the internal workings of the system 10. A control door 204 located on top of the mobile unit 15 gives the patient direct access to a power button 210 and a display 206 without having to tilt the mobile unit 15. The display 206 consists of a multi-touch capacitive screen employing both mutual capacitance and self capacitance methods of sensing user inputs. This allows user access to various features and controls, and displays the status of device parameters including a notification of scheduled changes of consumables and the battery power level. The display 206 is also used for direct programmer access to upgrade to the latest operating system and core software revisions or simply to reprogram the system 10 for a different patient. An I/O door 212 gives access to connections for the pulse oximeter 196, IR sensor 195, and cannula tube 26. A storage door 208 is used to store system related items such as spare consumables and a battery wall charger. A service access door 234 is formed by a concealed zipper located on the top perimeter of the mobile unit 15 under a fold in the polyester fabric 216. When the service access door 234 is unzipped, a technician can remove the aluminum frame skeleton 25, which securely houses all the internal workings making it easy to service the system 10.

The heart of the system 10 is the PC/104 computer module 218 along with a Digital Signal Processing (DSP) board 220 and an Analog/Digital, Input/Output data acquisition and control board 222. The PC/104 module 104 is an embedded form factor computer motherboard that functions as the overall operating system of the system 10 and provides data acquisition and control, harmonic analysis of the pulse oximeter output (DSP), algorithm computations for flow rate, temperature, humidity, and programming, including software changes and updates.

The PC/104 computer module 104 is connected to a wire harness as shown previously in, for example, FIGS. 5, 31, and 34, which connects to the Power Management Board or PMB 228. The PMB 228 is responsible for accepting battery power or power from a wall charger and delivering it to every electrically dependent item in the system 10, such as the blower 232, the display 206, the oximeter 196, and the computer module 218. The PMB 228 is also responsible for maintaining proper charging of the two 12-Volt DC lithium ion rechargeable batteries 40.

Consumables ensure a safe and clean system 10 without regular laborious cleaning procedures. Consumables are installed according to a recommended replacement schedule as prompted on the system display 106. The consumables include the lithium ion battery 40, the HEPA filter 32, the distilled water 52, a heater/venturi module 226, which is removable, replaceable, and unitary, and the cannula 26.

To use the system 10, a patient begins by placing the shoulder strap 100 over his or her 20 shoulder and continues by adjusting the strap 100 as necessary, connecting the waist snap buckle, adjusting as needed, connecting the ear oximeter 196, putting on the cannula 26, and turning the system on. System initialization measures air temperature and the patient's oxygen saturation and respiratory rate using data received from the plethysmogram wave form of the pulse oximeter 196 and performing harmonic analysis of continuous wavelet transforms, such as by using digital signal processing. This determines the initial setting of blower rate, humidity, and temperature settings and oxygen level.

Outside air enters through a HEPA filter 32. The filter 32 in this embodiment is a one piece design with a plastic rim and protective screen that easily snaps to the volute input 230 of the blower 232 creating a hermetic seal to ensure clean air. The DC powered variable-speed centrifugal blower 232 pushes the filtered air through its volute input 230 and into the reducing air chamber 46. Together, the blower 232 and reducing chamber 46 create compressed air by reducing the volume of air as it continues down the tube of the reducing air chamber 46 at a constant rate from additional air pushing from behind. Changes in air flow are made by varying the blower speed. The final output of air is a constant flow up to 40 lpm at a sustainable pressure of about 15 cm of H₂O.

This varying speed of the blower 232 operates in tandem with an oxygen digital control flow meter 76. The oxygen and compressed air combine in a mixing chamber 224. During inhalation, the oxygen flow meter 76 turns on while the air flow meter 78 decreases slightly to maintain constant volume of air entering the cannula 26. Turning on the oxygen flow only when it is needed during inspiration makes efficient use of the oxygen thereby making it viable to integrate an onboard liquid oxygen pulse dose system. After the mixing chamber 224, the air enters the venturi/heater module 226. This replaceable module 226 contains a venturi and heating element and is connected directly to the distilled water bottle 52 via a rubber stopper and needle connection for easy exchange of the distilled water consumable. The venturi and heating action humidify the air to 100% relative humidity and heat the oxygenated air up to 30° C. The heated and oxygenated air then enters the cannula tube and eventually enters into the nasal cannula 26. To control temperature and humidification, data is collected through the IR heat/humidification sensor 195 located within the upper portion of the shoulder strap 100 and returned to the data acquisition and control board 222. Continuous monitoring of system inputs keep the mobile unit 15 operating within comfortable, stable parameters and keeps pace with the ever changing needs of a patient as the patient changes in respiratory demand.

The battery 40 is a critical component in supporting the portability of the mobile unit 15. Several standard and unique options of charging can be offered, including a receptacle style wall charger, such as a transformer type, or an auto charger for connecting to the 12V charger receptacle of an automobile. In still other embodiments, a trickle charge can be gained by a human powered generator, such as a bicycle wheel generator. Even further, alternative energy can be exploited, such as through a flexible solar pack worn on the front or rear of a jacket with safety reflective tape similar to that worn by joggers. Each charging option allows the patient to use the mobile unit 15 even while the battery 40 is charging.

The mobile unit 15 may be used with an existing oxygen system. However, due to the ability of the system 10 to utilize oxygen very efficiently by monitoring respiratory rates and oxygen saturation levels, it will be appreciated that the system 10 can integrate an onboard liquid oxygen pulse dose system. This would free the patient from carrying an oxygen bottle at all times and would reduce oxygen expense by reducing oxygen usage.

Advantageously, the system 10 can be used as a medical data collection device. Since the system 10 is supported by a PC/104 computer module 218, that module 218 can readily include an onboard hard drive. This hard drive can continuously collect data from the patient and the data acquisition and control board 222 or otherwise. Data analysis can compute daily and weekly calories burned, such as by extrapolating from the system data and, possibly, by comparing the same to a physician's prescribed exercise schedule. This data can then be displayed on the touch screen display 206 aiding both the patient and physician in reaching the patient's target exercise goal. Additional medical data can also be downloaded for other medical reasons via wireless communication, such as communication operating under the wireless communication protocol referred to under the trademark BLUETOOTH of Bluetooth SIG, Inc., at a respiratory rehabilitation center. Wireless data transfer helps promote patient ease of use while stressing the portability and wireless feel of the mobile unit 15.

Still further, it is within the scope of the invention for the system 10 to be used for drug delivery. With the application of positive air pressure provided by the system 10, the patient's airways will tend to stent open thereby creating an ideal time for drug deposition. With the nasal cannula 26 already in use and the timing of inspiration known, drug deposition during the application of positive air pressure enables an ideal method for drug delivery employing the system 10.

With certain details and embodiments of the present invention disclosed, it will be appreciated by one skilled in the art that numerous changes and additions could be made thereto without deviating from the spirit or scope of the invention. This is particularly true when one bears in mind that the presently preferred embodiments merely exemplify the broader invention revealed herein. Accordingly, it will be clear that those with major features of the invention in mind could craft embodiments that incorporate those major features while not incorporating all of the features included in the preferred embodiments.

Therefore, the following claims are intended to define the scope of protection to be afforded to the inventor. Those claims shall be deemed to include equivalent constructions insofar as they do not depart from the spirit and scope of the invention. It must be further noted that a plurality of the following claims express certain elements as means for performing a specific function, at times without the recital of structure or material. As the law demands, these claims shall be construed to cover not only the corresponding structure and material expressly described in this specification but also all equivalents thereof.

1. A system for pulmonary rehabilitation by the application of positive airway pressure, the system comprising:
a source of pressurized air;
a source of pressurized oxygen;
control arrangement for enabling a control of the sources of pressurized air and pressurized oxygen;
a source of sterile water;
a source of heat; and
an output arrangement for providing pressurized and heated air and oxygen to a patient.

2. The system of claim 1 further comprising a mobile casing wherein the control arrangement, the source of sterile water, the source of heat, and the output arrangement are retained by the mobile casing.

3. The system of claim 2 wherein the mobile casing is chosen from the group consisting of a a waist pack, a backpack, a shoulder pack, and a rolling backpack.

4. The system of claim 1 further comprising a venturi chamber disposed to receive compressed air.

5. The system of claim 1 wherein the output arrangement comprises a nasal cannula.

6. The system of claim 5 wherein the output arrangement further comprises a weak link disconnect interposed along the output arrangement.

7. The system of claim 2 wherein the mobile casing has wheels disposed thereon for enabling a rolling of the mobile casing.

8. The system of claim 7 further comprising at least one secondary wheel rotatably coupled to the casing for providing secondary support to the casing wherein the secondary wheel is spaced form the wheels.

9. The system of claim 7 further comprising a means for enabling an automatic following of a patient by the casing.

10. The system of claim 9 wherein the means for enabling an automatic following of a patient comprises a tether.

11. The system of claim 10 wherein the means for enabling an automatic following of a patient further comprises a resilient member interposed along the tether.

12. The system of claim 9 wherein the means for enabling an automatic following of a patient comprises an automated “follow me” arrangement for enabling an automated following of a patient and a propulsion arrangement with a power source for propelling the casing to enable an automated following of a patient wherein the “follow me” arrangement comprises a “follow me” transceiver coupled to the casing and a “follow me” transponder for being retained by a patient.

13. The system of claim 7 further comprising a telescoping handle operably coupled to the casing.

14. The system of claim 1 further comprising an exercise device for enabling exercise of a patient during application of positive airway pressure and wherein the output arrangement comprises a nasal cannula.

15. A method for providing pulmonary rehabilitation by the application of positive airway pressure with the system of claim 14, the method comprising the steps of:
providing a system according to claim 14;
providing pressurized and heated air and oxygen to a patient through the output arrangement by retaining the nasal cannula in relation to the nostrils of the patient and employing the control arrangement to dispense pressurized air and pressurized oxygen through the nasal cannula; and
exercising by the patient by using the exercise device.

16. The method of claim 15 wherein the step of exercising by the patient using the exercise device is carried out while maintaining the mouth of the patient in a substantially closed position thereby to assist in imparting positive airway pressure to the patient.

17-23. (canceled)

24. A mobile system for pulmonary rehabilitation by the application of positive airway pressure, the mobile system comprising:
a mobile casing;
a compressor retained within the casing for compressing air to be provided to a user;
a means for retaining an oxygen tank in relation to the casing for providing a volume of compressed oxygen to a user;
an insulated heater retained within the casing for heating gasses to be provided to the user;
a means for retaining a power source relative to the mobile casing for providing power to the compressor and the heater;
a means for retaining a volume of sterile water relative to the casing;
a control arrangement retained relative to the casing;
an output arrangement comprising a nasal cannula for providing pressurized and heated air and oxygen to a user; and
a means for retaining the mobile casing relative to the user.

25. The mobile system of claim 24 wherein the means for retaining the mobile casing relative to the user comprises a strap with a hollow sleeve for receiving a portion of the nasal cannula.

26. The mobile system of claim 25 wherein the hollow sleeve of the strap is thermally insulated.

27. The mobile system of claim 25 wherein the hollow sleeve is accessible by a fastening arrangement disposed along substantially the entire hollow sleeve.

28. The mobile system of claim 25 further comprising a heat/humidity sensor retained relative to the strap and disposed adjacent to the nasal cannula for providing a measurement of heat and humidity of gasses passing through the nasal cannula.

29. The mobile system of claim 24 wherein the control arrangement includes a means for acquiring data and further comprising an oximeter ear clip lead for acquiring data regarding oxygen saturation of the user's blood.

30. The mobile system of claim 29 wherein the means for retaining the mobile casing relative to the user comprises a strap with a hollow sleeve for receiving at least a portion of the oximeter clip lead.

31. The mobile system of claim 24 wherein the control arrangement includes means for data acquisition and means for control of the compressor and insulated heater based on acquired data.

32. The mobile system of claim 31 wherein the control arrangement further includes a touch capacitive screen for enabling control and status display.

33. The mobile system of claim 24 wherein the control arrangement includes means for user data acquisition and means for data retention whereby data regarding a user can be collected over a period of time.

34. The mobile system of claim 24 further comprising a venturi disposed within the mobile casing for receiving gasses therethrough wherein the venturi and the heater are configured as a unitary, removable and replaceable module.

35. The mobile system of claim 24 wherein the compressor comprises a blower in combination with a reducing chamber.
